(19) United States
(12) Patent Application Publication
(10) Pub. No.: US 2002/0103466 A1
(43) Pub. Date: Aug. 1, 2002

(54) Sport nasal drip absorber

(76) Inventor: Stephen V. Petix JR., San Diego, CA (US)

Correspondence Address: Don E. Erickson, Law Offices, PMB 182, 3830 Valley Center Drive # 705, San Diego, CA, 92130-2331, US

(21) Appl. No.: 09/775,248

(22) Filed: Feb. 1, 2001

(51) Int. Cl. A61F 13/15

(52) U.S. Cl. 604/317; 604/327; 604/346; 604/358; 604/386; 604/387; 604/391; 2/159; 2/161.8; 2/163

(57) ABSTRACT

An apparatus for absorbing nasal drip during an activity, the apparatus consisting of a means for encapsulating at least one digit of a hand with a flexible, absorbent material. The apparatus is secured to the hand by means of a strap extending from the absorbent material that is secured about the wrist of the user. The apparatus may be sized to fit over a gloved hand to enable the use of the apparatus in different conditions.

The invention relates to nasal drip absorption means, and more particularly to an absorbent material that may be affixed to the hand of a user during an activity.

### BACKGROUND OF THE INVENTION

In various activities, such as in running, and in cold weather activities such as snow skiing, tobogganing, snowmobiling and the like, the nose tends to discharge fluid. However, when the person is involved in the activity, it may be impracticable, or inconvenient, to retrieve a handkerchief or tissue paper in order to relieve the dripping condition. This is particularly true during running activities, when the runner may not have any pockets for carrying a handkerchief for tissue, or.when the user is wearing gloves, a coat and other heavy apparel, and must remove his glove and sometimes open his coat in order to retrieve a handkerchief or tissue paper for absorbing the nasal drip.

There have been few attempts in the prior art to address this problem. U.S. Pat. No. 4,244,057 describes and claims an apparatus for attaching to an outer garment, such as a glove, of a user. The apparatus includes an absorbent material which than can be removably attached to the glove. Alternately, the apparatus could be attached to a wristband that fits over the glove around the wrist of the user. However, this prior art invention requires the apparatus be attached to the clothing of the user, and the apparatus suffers some of the same problems that it is supposed to address. To replace the removable tissue, the user must still remove the glove in order to obtain access pockets where replace tissues may be kept.

There therefore is a continuing need among pursuers of outdoor activities for some means of wiping the nose conveniently without requiring access to storage pockets. Such an item has heretofore been unavailable.

### SUMMARY OF THE INVENTION

The present invention describes an apparatus for absorbing nasal drip during various activities, the apparatus comprising means for encapsulating at least one digit of a Specification hand with an absorbent material. The absorbent material is maintained on the hand of the user by means of a connecting strap which wraps around the hand or wrist of the user, and secures the absorbent material by securing means such as a Velcro® material, snaps, or some other equivalent means.

It is therefore among the objects of the invention to provide a heretofore unavailable product for convenient absorption of nasal drip without the need to access a handkerchief or tissue. These and other objects, advantages and features of the invention will be apparent from the following description of the preferred embodiments, taken in conjunction with the accompanying drawings.

### DESCRIPTION OF THE DRAWINGS

FIG. 1 is a plan view of a first embodiment of an apparatus of the invention on the hand of a user.

FIG. 2(a) is a bottom view of the embodiment of FIG. 1 in an extended position.

FIG. 2(b) is a side view of the embodiment of FIG. 1 in an extended position.

FIG. 2(c) is a plan view of the embodiment of FIG. 1 in an extended position.

FIG. 3 is a plan view of a second embodiment of an apparatus of the invention on the hand of a user.

FIG. 4 is a plan view of a third embodiment of an apparatus of the invention on the hand of a user.

### DESCRIPTION OF THE PREFERRED EMBODIMENTS

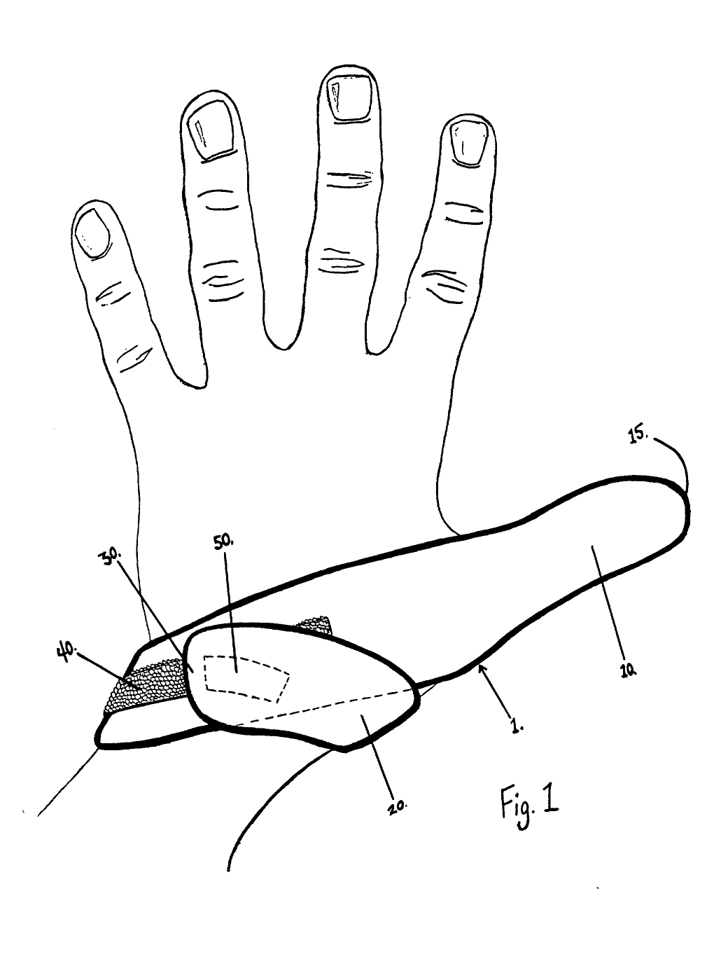

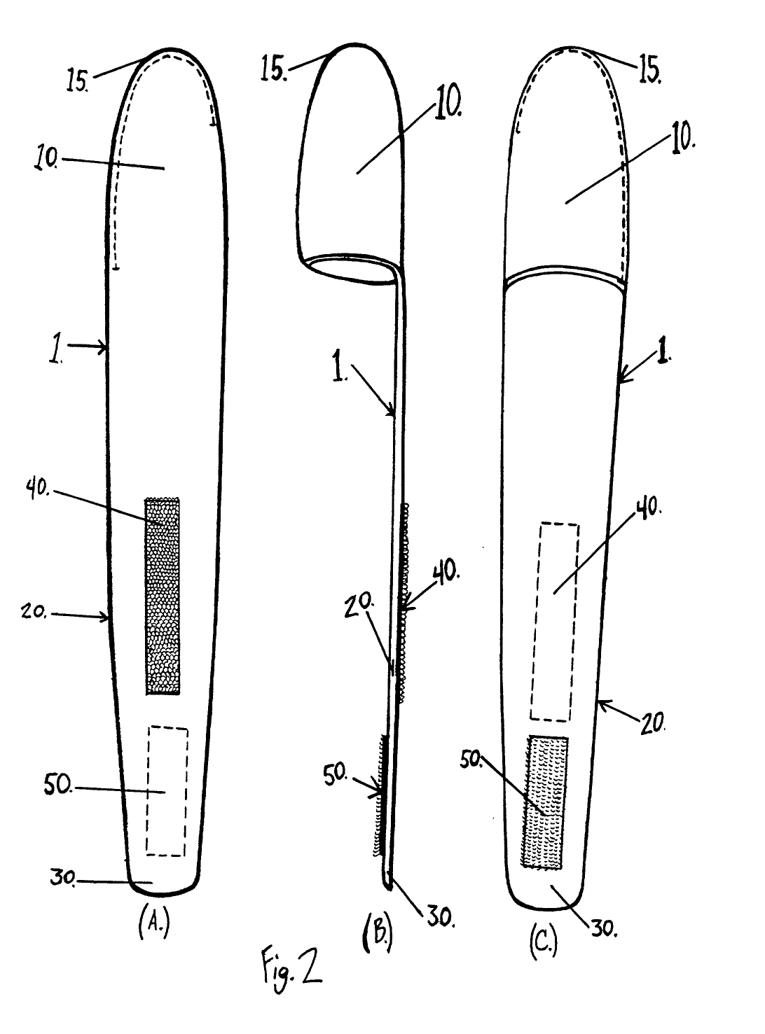

FIG. 1 depicts the nasal drip absorbent apparatus 1 on the hand of a user. FIGS. 2(a), (b), and (c) depict absorbent apparatus 1 in an extended position. Absorbent apparatus 1 is comprised of a somewhat cylindrical receptacle 10 for receiving at least one digit of a user's hand. It is immaterial on which digit of the user's hand absorbent apparatus 1 is placed, so long as it is convenient for the user to access his/her nose. Receptacle 10 may be sized to fit any digit or combination of digits. In many instances the user may wish receptacle 10 to be fitted over the thumb, and in other instances the Specification user may wish receptacle 10 to be fitted over the first, or first and second, finger(s). In the preferred embodiment receptacle 10 is fabricated to have a closed end 15, however, receptacle 10 may be constructed to be open-ended, forming a sleeve-like receptacle, thereby enabling receptacle 10 to be adapted to any length digit.

Extending from receptacle 10 is a wrist strap 20, having at its distal end a means of securing strap 20 about the wrist of the user, thereby securing receptacle 10 on the selected digit of the hand, and thereby enabling the user to wipe nasal drip from his/her nose. Absorbent apparatus 1 may be sized to fit over the glove of a user, thereby requiring a circumferentially larger receptacle 10, and a longer wrist strap 20, to enable absorbent apparatus to be secured around the gloved wrist of the user.

Absorbent apparatus 1 may be fabricated from one single piece of material, or it may be fabricated from two different materials. The method a fabrication will depend on the usage of absorbent apparatus 1 and/or the strength characteristics desired. For example, a jogger or runner may desire a light-weight absorbent apparatus, without regard to the strength requirements. In such case, it may be desirable to fabricate absorbent apparatus 1 from a single piece of material such as described in U.S. Pat. No. 4,502,156, an improved, non-woven polypropylene fabric which displays excellent wetting characteristics for transferring moisture from a moisture-bearing surface utilizing the fabric. This material and similar textiles are washable, providing for a non-disposable tissue type apparatus.

On the other hand, a skier may require an absorbent apparatus 1 with more demanding strength characteristics where the apparatus may be abraded from using a ski pole, and similarly, a snowmobile operator may be gripping a steering wheel that may abrade absorbent apparatus 1. Therefore, it may be desirable to fabricate absorbent apparatus 1 from an abrasion resistant material, such as a high strength nylon or leather, and then attach, such as by sewing, gluing, or other means, the moisture absorbing material to receptacle 10. Alternatively, manufacturers of outdoor clothing are constantly improving textiles that demonstrate superior wicking and wetting characteristics. Such materials may also demonstrate greater strength characteristics, and may be selected in Specification fabricating absorbent apparatus to form a single, higher-strength absorbent apparatus.

Referring again to FIGS. 1 and 2(a), wrist strap 20 is sized to circumferentially encapsulate the wrist of the user and may be secured about the wrist by numerous conventional fasteners. For example, absorbent apparatus 1 of the preferred embodiment is secured by means of VELCRO® strips, i.e., a complementary pair of strips consisting of one strip of fabric of small hooks that sticks to a corresponding strip of small loops, and wherein median strip 40 is located at the approximate longitudinal midpoint of wrist strap 20 (FIG. 2(b)), and complementary distal strip 50 is located at the distal end of wrist strap 20 (FIG. 2(c)). Median strip 40 may be of any selected length such that it provides the capacity of wrist strap 20 being of adjustable circumference about the wrist of the user, such that when receptacle 10 is fitted over the selected digit of the user, and wrist strap 20 is wrapped about the wrist of the user, distal strip 50 adjustable engages median strip 40, thereby securing absorbing apparatus 1 to the hand of the user.

The absorbent apparatus of the present invention is not limited by the means of securing the apparatus to the users wrist. Alternatively, as shown in FIG. 3, wrist strap 20 could be fabricated of an elastic material forming a loop 70, and sized to expand to pass over the hand and which contracts sufficiently about the user's wrist to secure absorbing apparatus 1 to the user's hand.

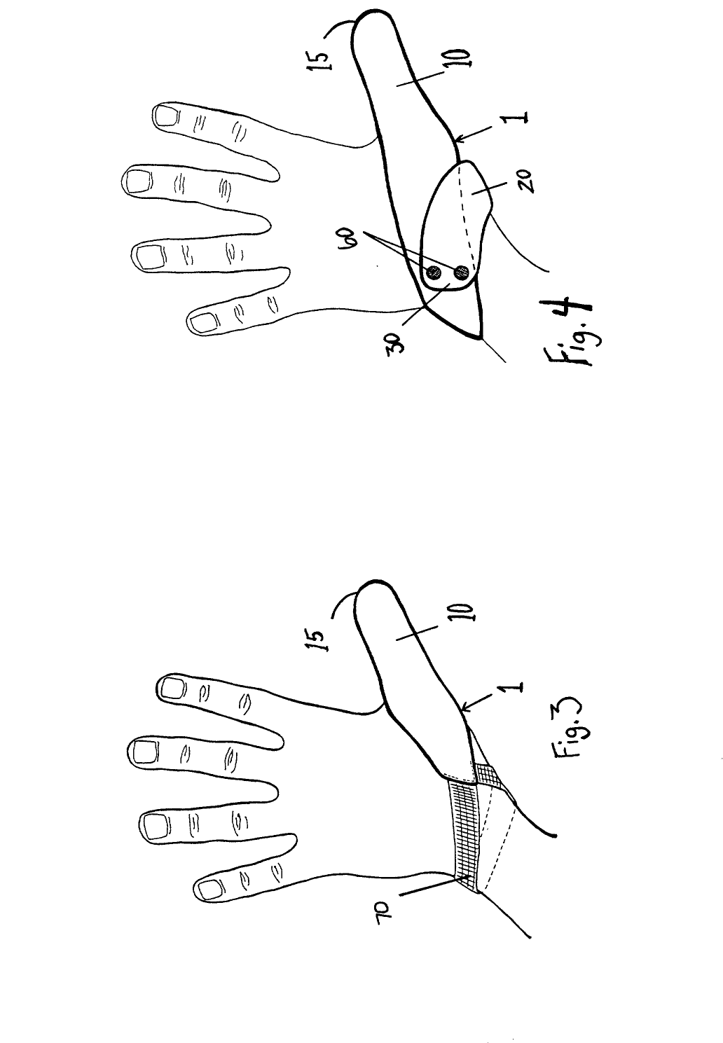

Alternatively, as shown in FIG. 4, the apparatus could be secured to the user's wrist by the means of snaps, wherein at least one complementary part of the snap 60 attached to wrist strap 30 at a selected location at the approximate longitudinal midpoint of wrist strap 30, and the opposing complementary part of a snap 60 attached at distal end of wrist strap 30 such that when wrist strap 30 is wrapped about the wrist of the user, the distal end snap engages median snap, thereby securing absorbing apparatus 1 to the hand of the user.

While the present description contain many specificities, these should not be construed as limitations on the scope of the invention, but rather as an exemplification of some preferred embodiments thereof.

I claim:

1. An apparatus for absorbing nasal drip during an activity, the apparatus comprising means for encapsulating at least one digit of a hand, the encapsulating means having an absorbing end and a securing end, the absorbing end for receiving the at least one digit of the hand, the securing end having means for securing the apparatus about the hand; and
wherein the absorbing end comprises at least one layer of flexible absorbent material.

2. The apparatus of claim 1 wherein the absorbing end is sized to encapsulate a thumb.

3. The apparatus of claim 1 wherein the absorbing end is sized to encapsulate at least one digit of the hand while the hand is encased in a glove.

4. The apparatus of claim 1 wherein the flexible absorbent material is fabricated from a fabric demonstrating wetting characteristics of transferring moisture from a moisture-bearing surface.

5. The apparatus of claim 4 wherein the flexible absorbent material is fabricated from a polypropylene fabric.

6. The apparatus of claim 1 wherein the flexible absorbent material is washable.

7. The apparatus of claim 1 wherein the securing end is substantially planar and sized to extend and overlap about a wrist of the user.

8. The apparatus of claim 7 wherein the means for securing the apparatus claims about the hand comprises VELCRO® type material.

9. The apparatus of claim 7 wherein the means for securing the apparatus about the hand comprises a complementary snap device affixed to the securing end.

10. The apparatus of claim 7 wherein the means for securing the apparatus about the hand comprises an elastic material affixed to the securing end.

11. The apparatus of claim 1 wherein the means for securing the apparatus about the hand comprises is fabricated from the same material as is the flexible absorbent material.

12. An apparatus for absorbing nasal drip during an activity, the apparatus comprising means for encapsulating at least one digit of a hand, the encapsulating means having an absorbing end and a securing end, the absorbing end for receiving the at least one digit of the hand, the securing end having means for securing the apparatus about the hand, the securing end being substantially planar and sized to extend and overlap about a wrist of the user; and
wherein the absorbing end comprises at least one layer of flexible absorbent material.

13. The apparatus of claim 12 wherein the absorbing end is sized to encapsulate a thumb.

14. The apparatus of claim 12 wherein the absorbing end is sized to encapsulate a digit of the hand while the hand is encased in a glove.

15. The apparatus of claim 12 wherein the flexible absorbent material is fabricated from a fabric demonstrating wetting characteristics of transferring moisture from a moisture-bearing surface.

16. The apparatus of claim 15 wherein the flexible absorbent material is fabricated from a polypropylene fabric.

17. The apparatus of claim 12 wherein the flexible absorbent material is washable.

18. The apparatus of claim 12 wherein the means for securing the apparatus about the hand comprises VELCRO® type material.

19. The apparatus of claim 12 wherein the means for securing the apparatus about the hand comprises a complementary snap device affixed to the securing end.

20. The apparatus of claim 12 wherein the means for securing the apparatus about the hand comprises an elastic material affixed to the securing end.

21. The apparatus of claim 12 wherein the means for securing the apparatus about the hand comprises is fabricated from the same material as is the flexible absorbent material.

22. An apparatus for absorbing nasal drip during an activity, the apparatus comprising means for encapsulating at least one digit of a hand, the encapsulating means having an absorbing end and a securing end, and wherein:
(a) the absorbing end, for receiving the at least one digit of the hand, comprises at least one layer of flexible absorbent material, the flexible absorbent material fabricated from a fabric demonstrating wetting characteristics of transferring moisture from a moisture-bearing surface; and
(b) the securing end having means for securing the apparatus about the hand, the securing end being substantially planar and sized to extend and overlap about a wrist of claims the user.

23. The apparatus of claim 22 wherein the absorbing end is sized to encapsulate a thumb.

24. The apparatus of claim 22 wherein the absorbing end is sized to encapsulate a digit of the hand while the hand is encased in a glove.

25. The apparatus of claim 22 wherein the flexible absorbent material is fabricated from a polypropylene fabric.

26. The apparatus of claim 22 wherein the flexible absorbent material is washable.

27. The apparatus of claim 22 wherein the means for securing the apparatus about the hand comprises VELCRO® type material.

28. The apparatus of claim 22 wherein the means for securing the apparatus about the hand comprises a complementary snap device affixed to the securing end.

29. The apparatus of claim 22 wherein the means for securing the apparatus about the hand comprises an elastic material affixed to the securing end.

30. The apparatus of claim 22 wherein the means for securing the apparatus about the hand comprises is fabricated from the same material as is the flexible absorbent material.
